(19) United States
(12) Patent Application Publication
(10) Pub. No.: US 2004/0013731 A1
(43) Pub. Date: Jan. 22, 2004

(54) Drug-complex microparticles and methods of making/using same

(75) Inventors: Li-Lan H. Chen, Edison, NJ (US); Alfred Liang, Edison, NJ (US); Xu Zheng, Edison, NJ (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Lavipharm Laboratories Inc.

(21) Appl. No.: 10/409,696

(22) Filed: Apr. 8, 2003

Related U.S. Application Data
(60) Provisional application No. 60/371,218, filed on Apr. 8, 2002.

(51) Int. Cl. A61K 31/473; A61K 9/14

(52) U.S. Cl. 424/486; 424/487; 514/290

(57) ABSTRACT

Oral drug complex microparticles containing: a pharmaceutical active having a nitrogen moiety and an anionic methacrylic acid copolymer; wherein the oral drug complex micropaticles resist dissolution or dissociation upon exposure to saliva, but which rapidly dissociate in gastric acid and which rapidly dissolve in intestinal fluid. Oral drug delivery devices containing said oral drug complex microparticles are provided. Processes for producing said oral drug complex microparticles and methods for treating patients including administering said oral drug delivery devices thereto are also provided.

The present invention is directed to oral drug complex microparticles containing: a pharmaceutical active having a nitrogen moiety and an anionic methacrylic acid copolymer; wherein the oral drug complex micropaticles resist dissolution or dissociation upon exposure to saliva, but which rapidly dissociate in gastric acid and which rapidly dissolve in intestinal fluid. The present invention is also directed to oral drug delivery devices containing said oral drug complex microparticles; processes for producing said oral drug complex microparticles and methods for treating patients including administering said oral drug delivery devices thereto.

Many pharmaceuticals are administered orally using solid, shaped dosage forms such as tablets, pills and capsules that retain their shape under moderate pressure. Generally these dosage forms are designed to be swallowed. Accordingly, the disagreeable tastes associated with many pharmaceuticals delivered orally are often not of significant concern during formulation development of the dosage forms. The exception being provision for means to prevent the taste of these pharmaceuticals from being apparent to the patient during the short time that the dosage form is in the oral cavity. Such means may include the provision of an appropriately thin and quickly dissolving coating on the tablet, the use of capsules or simply compressing a tablet firmly so that it will not significantly disintegrate in the mouth.

Some patients, particularly pediatric and geriatric patients, experience difficulty swallowing or chewing solid dosage forms. Many pediatric and geriatric patients are unwilling to take such solid preparations, complaining that the drug is difficult to swallow or stops in the pharynx or gullet. To accommodate these patients, pharmaceutical actives are sometimes provided as powders or granules. Notwithstanding, these powders and granules may also be difficult to swallow because of their aptness to remain in the oral cavity and therefore to cause an unpleasant feeling in the mouth. In some instances, patients may choke when administered powders or feel a pain or unpleasantness due to granules lodging between false teeth and the pallet. In addition, powders and granules are often packaged in individual dose quantities in packages which some patients have difficulty opening. Liquid, syrups or suspensions are considered as desirable dosage forms for pediatric and geriatric patients. However, these dosage forms are often difficult to distribute, measure, dose and store due to the nature of dosage forms and problems with stability.

In the effort of assisting these patients to take their medication and ensure patient compliance, several fast-dissolving drug delivery systems, such as fast melt tablets, effervescent tablets, lyophilized wafers and paper wafers etc, have been developed. Fast dissolving drug delivery system can be manufactured by a variety of technologies, including direct compression, wet granulation, freeze drying and solvent coating.

The production of a palatable dosage form is very important for patient compliance. In the pharmaceutical industry, one of the preformulation tests performed on a new chemical entity is to determine its organoleptic properties, patient compliance, marketing and sale of the resulting products. If a drug is found to have unpleasant taste during the formulation study, several techniques can be considered for masking the unpleasant taste of the drug. Depending on the physicochemical properties of drugs, there are several approaches to prepare good-tasting or acceptable products and to minimize the bad tastes of drugs when the dosage form is chewed, disintegrated and/or dissolved in the oral cavity. These include (1) blending, (2) overshadowing, (3) physical methods, (4) chemical methods, and (5) physiological methods.

The most desired attributes of chewable tablets and fast-dissolving dosage forms are good taste and mouth feel. The dosage form should be bitterless, smooth and cooling in the mouth. The masking of unpleasant tastes is therefore an important consideration in the formulation of many therapeutic agents and can be achieved by several physical methods, barrier and complexation approaches, which are to minimize direct contact between the actives and the taste receptors in the oral cavity.

Barrier approaches to mask the taste of unpalatable drugs involve preventing the drugs from contacting the taste buds by coating or encapsulating the drugs with inert materials such as sugars, natural or synthetic polymers, lipids or waxes. Microencapsulation of drug particles by polymers, lipids or waxes is the most common taste masking technique for chewable tablets and some fast dissolving dosage forms. Several methods and materials, which are well known in the art, have been used to coat drug particles and granules.

Taste masking of bitter anionic and cationic drugs can be accomplished by complexing the drugs with either cation or anion exchange resins, respectively. Such approaches have been used in pharmaceutical industry in the effort of making bitterless active composition and palatable oral products. Neutral drugs can be adsorbed onto adsorbents such as silicon dioxide, magnesium trisilicate or magnesium aluminum silicate.

### SUMMARY OF THE INVENTION

In a preferred embodiment of the present invention, oral drug complex microparticles are provided, containing: a pharmaceutical active having a nitrogen moiety and an anionic polymer containing acidic-reacting groups such as carboxylic, more preferably an anionic methacrylic acid copolymer such as Eudragit L 100-55, Eudragit L30 D-55, Eudragit L 100, Eudragit S 100, commercially available from Rohm America Inc, most preferably Eudragit L 100, most preferably an anionic methacrylic acid copolymer; wherein the oral drug complex micropaticles resist dissolution or dissociation upon exposure to saliva, but which rapidly dissociate in gastric acid and which rapidly dissolve in intestinal fluid. In a preferred aspect of this embodiment, the oral drug complex microparticles exhibit an ionic strength of less than 102 mEq/liter, more preferrably less than 70 mEq/liter.

In a preferred aspect of the present invention, the oral drug complex microparticles contain a pharmaceutical active having a nitrogen moiety selected from the group of acyclic amine, heterocyclic amine, amide, imine, imide and nitrile.

In another preferred aspect of the present invention, the oral drug complex microparticles contain a pharmaceutical active selected from the group of Famotidine and Loratidine.

In another preferred aspect of the present invention, the oral drug complex microparticles contain a pharmaceutical active having a nitrogen moiety and an anionic methacrylic acid copolymer, wherein pharmaceutical active and the anionic methacrylic acid copolymer are present in the oral drug complex microparticles in a ratio of 10:1 to 1:10, more preferably in a ratio of 3:1 to 1:3; most preferably in a ratio of 1:1.

In another preferred aspect of the present invention, the oral drug complex microparticles have a particle size of less than 50 μm; more preferably a particle size of less than 25 μm.

The oral drug complex microparticles of the present invention may effectively mask the unpleasant tastes associated with many pharmaceutical actives and be incorporated into a variety of drug delivery devices including powders, chewable tablets, tablets, fast melt sugar tablets, lypholized wafers, quick dissolving paper wafers, mucoadhesive oral drug delivery films and non-mucoadhesive oral drug delivery films.

In another preferred embodiment of the present invention, oral drug delivery devices are provided containing oral drug complex microparticles, containing: a pharmaceutical active having a nitrogen moiety and an anionic polymer containing acidic-reacting groups such as carboxylic, more preferably an anionic methacrylic acid copolymer such as Eudragit L 100-55, Eudragit L30 D-55, Eudragit L 100, Eudragit S 100, commercially available from Rohm America Inc, most preferably Eudragit L 100, most preferably an anionic methacrylic acid copolymer; wherein the oral drug complex micropaticles resist dissolution or dissociation upon exposure to saliva, but which rapidly dissociate in gastric acid and which rapidly dissolve in intestinal fluid. In a preferred aspect of this embodiment, the oral drug complex microparticles exhibit an ionic strength of less than 102 mEq/liter, more preferrably less than 70 mEq/liter.

In a preferred aspect of the present invention, the oral drug delivery devices are provided in the form of a powder, a chewable tablet, a tablet, a fast melt sugar tablet, a lyophilized wafer, an intraoral paper wafer, a mucoadhesive film and a non-mucoadhesive film.

In another preferred aspect of the present invention, the oral drug delivery devices may further contain stabilizers, taste modifying agents, preservatives, coloring agents, surfactant/wetting agents, plasticizers and water soluble film formers.

In another preferred aspect of the present invention, the oral drug delivery devices may further contain a buffer, most preferrably a sodium bicarbonate buffer.

In another preferred aspect of the present invention, the oral drug complex microparticles contain a pharmaceutical active having a nitrogen moiety selected from the group of acyclic amine, heterocyclic amine, amide, imine, imide and nitrile.

In another preferred aspect of the present invention, the oral drug complex microparticles contain a pharmaceutical active selected from the group of Famotidine and Loratidine.

In another preferred aspect of the present invention, the oral drug complex microparticles contain a pharmaceutical active having a nitrogen moiety and an anionic polymer containing acidic-reacting groups such as carboxylic, more preferably an anionic methacrylic acid copolymer such as Eudragit L 100-55, Eudragit L30 D-55, Eudragit L 100, Eudragit S 100, commercially available from Rohm America Inc, most preferably Eudragit L 100, most preferably an anionic methacrylic acid copolymer, wherein pharmaceutical active and the anionic polymer are present in the oral drug complex microparticles in a ratio of 10:1 to 1:10, more preferably in a ratio of 3:1 to 1:3; most preferably in a ratio of 1:1.

In another preferred aspect of the present invention, the oral drug complex microparticles have a particle size of less than 50 μm; more preferably a particle size of less than 25 μm. The oral drug complex microparticles of the present invention preferably are water insoluble, bitterless and tasteless in the oral cavity, dissociate upon exposure to gastric fluids and dissolve in intestinal fluids.

In another preferred embodiment of the present invention, a process for producing oral drug complex microparticles is provided, including: (a) dissolving an anionic polymer, preferably a polymethylacrylate copolymer, in an alcohol or a hydro-alcoholic solution; (b) dissolving or suspending a pharmaceutical agent having a nitrogen moiety in a buffer solution; (c) simultaneously feeding the products of (a) and (b) into a high energy ultrasonic processor to produce a complex solution; (d) discharging the complex solution to a spray dryer for drying; and, (e) collecting the product oral drug complex microparticles. The process of the present invention preferably results in very limited to no degradation of the pharmaceutical active being processed.

In a preferred aspect of the present invention, products of (a) and (b) are exposed to ultrasonic energy in (c) having a frequency between 25 and 40 kHz until a clear complex solution is produced.

In another preferred aspect of the present invention, the ultrasonic energy may be generated by at least one of mechanical action and electromechanical action.

In another preferred embodiment of the present invention, a method for treating a patient is provided including administering to the patient an oral drug delivery device containing oral drug complex microparticles, containing: a pharmaceutical active having a nitrogen moiety and an anionic polymer containing acidic-reacting groups such as carboxylic, more preferably an anionic methacrylic acid copolymer such as Eudragit L 100-55, Eudragit L30 D-55, Eudragit L 100, Eudragit S 100, commercially available from Rohm America Inc, most preferably Eudragit L 100, preferably an anionic methacrylic acid copolymer; wherein the oral drug complex micropaticles resist dissolution or dissociation upon exposure to saliva, but which rapidly dissociate in gastric acid and which rapidly dissolve in intestinal fluid. In a preferred aspect of this embodiment, the oral drug complex microparticles exhibit an ionic strength of less than 102 mEq/liter, more preferrably less than 70 mEq/liter.

### BRIEF DESCRIPTION OF THE DRAWING

There are shown in the drawings certain exemplary embodiments of the present invention as presently preferred. It should be understood that the present invention is not limited to the embodiments disclosed as examples, and is capable of variation within the spirit and scope of the appended claims.

In the drawings,

FIG. 1 depicts the chemical structure of methyl methacrylate copolymers,

FIG. 2 depicts the ionic interation of acidic polymer (Eudragit L100) and amine drugs,

FIG. 3 depicts a preferred process of the invention for producing acidic polymer-basic drug microparticles,

FIG. 4 is a graphical representation of the effect of pH on a preferred famotidine-polymer (Eudragit L100) complex of present invention,

FIG. 5 is a graphical representation of the dissolution profiles in artificial saliva of the Famotidine containing oral drug complex microparticles discussed in Examples 1-3,

FIG. 6 is a graphical representation of the dissociation profiles in gastric fluid of the Famotidine containing oral drug complex microparticles discussed in Examples 1-3,

FIG. 7 is a graphical representation of the dissolution profiles in intestinal fluid of the Famotidine containing oral drug complex microparticles discussed in Examples 1-3, and

FIG. 8 is a graphical representation of the stability of some oral drug delivery devices of the present invention in the form of quick dissolving intraoral dosage films containing oral drug complex microparticles produced in accordance with Examples 1-3 herein.

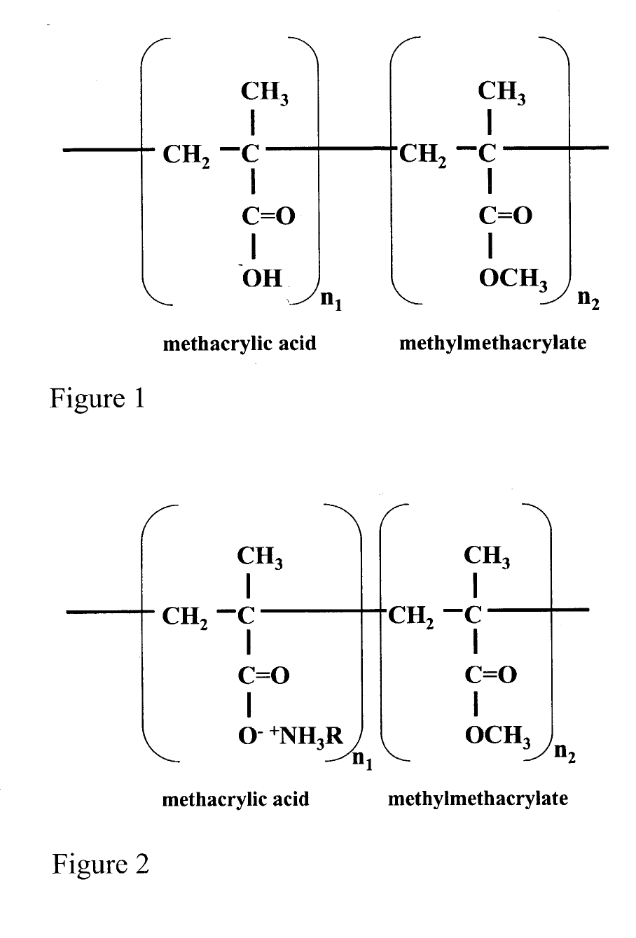

### DETAILED DESCRIPTION

The term “patient” as used herein and in the appended claims is an animal, preferably a mammal, more preferably a human.

The products and methods of the present invention provide a means for increasing the palatability of oral dosage forms. By increasing the palatability of such oral dosage forms, it is believed that patient compliance may be improved.

The present invention provides oral drug complex microparticles for use in oral drug delivery systems. The oral drug complex microparticles provided by the present invention, contain: a pharmaceutical active having a nitrogen moiety and an anionic polymer. The oral drug complex microparticles of the present invention preferably resist dissolution or dissociation upon exposure to saliva. The oral drug complex microparticles of the present invention, however, preferably, rapidly dissociate in gastric acid and rapidly dissolve in intestinal fluid. Preferably, the oral drug complex microparticles of the present invention may exhibit an ionic strength of less than 102 mEq/liter, more preferrably less than 70 mEq/liter.

Pharmaceutical actives suitable for use with the present invention include active agents having a nitrogen moiety selected from the group of acyclic amine, heterocyclic amine, amide, imine, imide and nitrile. More preferably, the pharmaceutical active may be selected from the group of Famotidine and Loratidine, most preferably Famotidine.

Anionic polymers suitable for use with the present invention include anionic polymers containing acidic-reacting groups such as carboxylic, more preferably an anionic methacrylic acid copolymer such as Eudragit L 100-55, Eudragit L30 D-55, Eudragit L 100, Eudragit S 100, commercially available from Rohm America Inc, most preferably Eudragit L 100, most preferably an anionic methacrylic acid copolymer.

The ratio of pharmaceutical active to anionic polymer in the oral drug complex micropaticles of the present invention may be in a ratio range of 10:1 to 1:10, more preferably in a ratio range of 3:1 to 1:3; most preferably in a ratio range of 1:1.

The oral drug complex microparticles of the present invention preferably exhibit a particle size of less than 50 μm; more preferably a particle size of less than 25 μm.

The present invention also provides oral drug delivery devices which contain oral drug complex microparticles of the present invention. Drug delivery devices suitable for use with the present invention include conventional oral delivery devices. For example, oral delivery devices suitable for use with the present invention include powders, chewable tablets, oral tablets, fast melt sugar tablets, lyophilized wafers, intraoral paper wafers, mucoadhesive films and a non-mucoadhesive films, most preferably mucoadhesive and non-mucoadhesive films.

The oral drug delivery devices of the present invention may optionally contain buffers, stabilizers, taste modifying agents, preservatives, coloring agents, surfactant/wetting agents, plasticizers and water soluble film formers.

Buffers suitable for use with the present invention include, but are by no means limited to, citric acid, fumaric acid, lactic acid, tartaric acid, malic acid, sodium citrate, sodium bicarbonate, sodium carbonate, sodium phosphate, potassium phosphate and magnesium oxide, more preferably carbonated buffers, most preferably sodium bicarbonate buffers.

Stabilizers suitable for use with the present invention include, but are by no means limited to, anti-oxidants, chelating agents and enzyme inhibitors. Preferred stabilizers include ascorbic acid, vitamin E, butylated hydroxyanisole (BHA), butylated hydroxytoluene (BHT), propyl gallate, dilauryl thiodipropionate, thiodipropionic acid, gum guaiac, citric acid, edetic acid and its salts and glutathione.

Taste modifying agents suitable for use with the present invention include, but are by no means limited to, flavoring agents, sweetening agents and taste masking agents. Preferred taste modifying agents include the essential oils or water soluble extracts of menthol, wintergreen, peppermint, sweet mint, spearmint, vanillin, cherry, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durean, green tea, grapefruit, banana, butter, camomile, sugar, dextrose, lactose, mannitol, sucrose, xylitol, malitol, acesulfame potassium, talin, glycyrrhizin, sucralose, aspartame, saccharin, sodium saccharin, sodium cyclamate and honey.

Preservatives suitable for use with the present invention include, but are by no means limited to, anti-microbial agents and non-organic compounds. Preferred preservatives include sodium benzoate, parabens and derivatives, sorbic acid and its salts, propionic acids and its salts, sulfur dioxide and sulfites, acetic acid and acetates, nitrites and nitrates.

Coloring agents suitable for use with the present invention include, but are by no means limited to, FD & C coloring agents, natural coloring agents, natural juice concentrates and pigments. Preferred pigments include titanium oxide, silicon dioxide and zinc oxide.

Surfactant/wetting agents suitable for use with the present invention include, but are by no means limited to, poloxamer, polyoxyethylene alkyl ethers, polyoxyethylene castor oil derivatives, polyoxyethylene sorbitan fatty acid esters, polyoxyethylene stearates, sodium lauryl sulfate. Preferred surfactant/wetting agents include polyoxyethylene castor oil derivatives.

Plasticizing agents suitable for use with the present invention include, but are by no means limited to, glycerin, sorbitol, propylene glycol, polyethylene glycol, triacetin, triethyl citrate (TEC), acetyl triethyl citrate (ATEC) and other citrate esters.

Water soluble film formers suitable for use with the present invention include, but are by no means limited to, hydrocolloids, for example:

(a) water soluble non-gelling (at room temperature) natural polysaccharide or derivatives including pectin and derivatives, guar gum arabic, tragacanth gum, xanthan gum, gellan sodium salt, propyleneglycol alginate, starches (amylose, amylopectin), modified starches, hydroxyethyl starch, pullulan, carboxymethyl starch, gum ghatti, okra gum, karaya gum, dextrans, dextrins and maltodectrins, konjac, acemannan from aloe, locust bean gum, tara gum, quince seed gum, fenugreek seed gum, scleraglucan, gum arabic, psyllium seed gum, tamarind gum, oat gum, carrageenans, succinoglucan, larch arabinogalactan, flaxseed gum, chondroitin sulfates, hyaluronic acid, curdlan, chitosan, deacetylated konjac and rhizobium gum;

(b) water soluble non-gelling polypeptide or protein exemplified by gelatins, albumins, milk proteins, soy protein and whey proteins; and,

(c) synthetic hydrocolloids exemplified by polyethylene-imine, hydroxyethyl cellulose, sodium carboxymethyl cellulose, carboxymethyl cellulose, hydroxypropyl cellulose, sodium carboxymethyl cellulose, methyl cellulose, ethyl cellulose, polyacrylic acids, low molecular weight polyacrylamides and their sodium salts (carbomers), polyvinylpyrollidone, polyethylene glycols, polyethylene oxides, polyvinyl alcohols, pluronics, tetronics, and other block co-polymers, carboxyvinyl polymers, and colloidal silicon dioxide.

Preferred water soluble film formers include hydroxypropyl methyl cellulose having a methoxy content of 19-30% and a hydroxypropyl content of 7 to 12% with a molecular weight of 50,000 to 250,000 daltons.

The present invention also provides a process for producing the oral drug complex microparticles of the present invention. The process of the present invention includes (a) dissolving an anionic polymer, preferably a polymethylacrylate copolymer, in an alcohol or a hydro-alcoholic solution; (b) dissolving or suspending a pharmaceutical agent having a nitrogen moiety in a buffer solution; (c) simultaneously feeding the products of (a) and (b) into a high energy ultrasonic processor to produce a complex solution; (d) discharging the complex solution to a spray dryer for drying; and, (e) collecting the product oral drug complex microparticles.

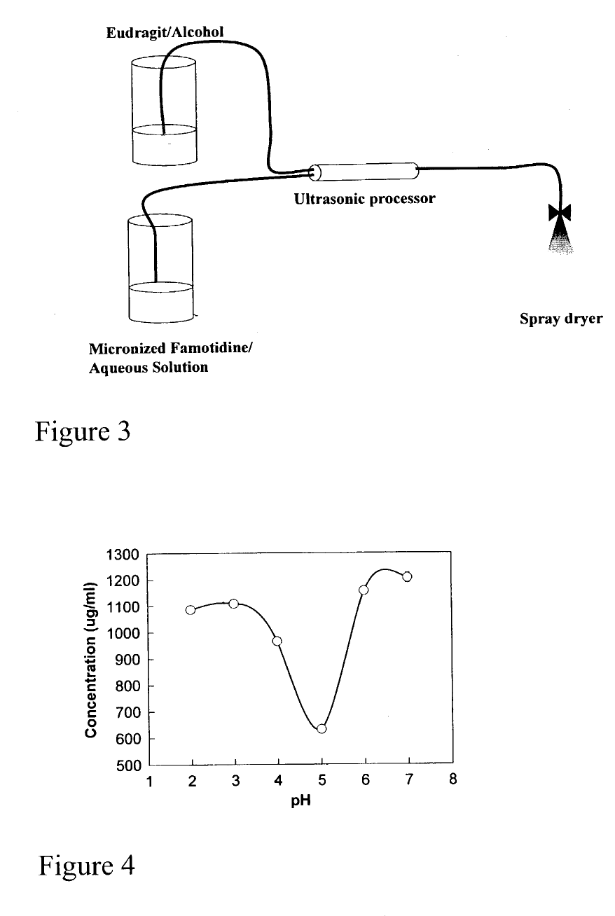

FIG. 3 provides an illustration of a preferred embodiment of the process of the present invention wherein the anionic polymer (Eudragit L-100 commercially available from Rohm America, Inc.) is dissolved or dispersed in alcohol, the pharmaceutical active is Famotidine which is dissolved or dispersed in aqueous solution.

During the process of the present invention, the products of (a) and (b) are preferably exposed to ultrasonic energy in (c) having a frequency between 25 and 40 kHz until a clear complex solution is produced.

The ultrasonic energy to which the products of (a) and (b) are subjected may preferably be generated using any conventional means, more preferably any conventional means using mechanical action or electromechanical action.

The present invention also provides a method for treating patients including administering to the patient an oral drug delivery device containing oral drug complex microparticles of the present invention.

### EXAMPLES

The preferred embodiments of the present invention will now be further described through the following examples set forth hereinbelow which are intended to be illustrative of the preferred embodiments of the present invention and are not intended to limit the scope of the invention as set forth in the appended claims.

### Examples 1-4

Oral drug complex microparticles of the present invention were prepared having the formulations indicated in Table 1 according to the processes discussed in the following examples.

| | Ex. 1 | Ex. 2 | Ex. 3 | Ex. 4 |
|---|---|---|---|---|
| Material | (in wt %) | (in wt %) | (in wt %) | (in wt %) |
| Famotidine | 18.7 | 17.0 | 16.0 | — |
| Loratidine | — | — | — | 70.0 |
| Methacrylic acid-methyl | 37.5 | 34.1 | 31.8 | 34.1 |
| methacrylate copolymer | | | | |
| (Eudragit L-100) | | | | |
| Sodium bicarbonate | 0.6 | 0.5 | 9.0 | .05 |
| Propylene glycol | 21.1 | — | 21.1 | — |
| Nipagin M/Nipasol M | 0.1 | 0.1 | 0.1 | 0.1 |
| Methocel E15 | 15.1 | 9.6 | 15.1 | 9.6 |
| Aspartame | 1.3 | 0.9 | 1.3 | 0.9 |
| Sunnett | 1.4 | 0.9 | 1.4 | 0.9 |
| Peppermint | 1.5 | 1.0 | 1.5 | 1.0 |
| Strawberry twist | 1.6 | 1.1 | 1.6 | 1.1 |
| Cremophore | 0.4 | 0.2 | 0.4 | 1.2 |
| Sodium EDTA | 0.2 | 0.1 | 0.2 | 0.1 |
| FD&C Red 40 | qs | qs | qs | qs |
| Sorbitol | — | 34.1 | — | 34.1 |
| Neophesperidine | — | 0.3 | — | 0.3 |

### Example 1

An oral drug delivery device comprising bitterless Famotidine oral drug complex microparticals incorporated into a mucoadhesive intraoral film was prepared as follows:

(1) the polymethylacrylate copolymer was dissolved in ethanol;

(2) the Famotidine was suspended in sodium bicarbonated;

(3) the products of (1) and (2) were simultaneously pumped into a high energy ultrasonic processor, producing a complex solution in situ;

(4) the product of (3) was discharged to a spray dryer for drying, i.e. removal of the ethanol;

(5) the product of (4) was free-flowing, bitterless drug-polymer complex microparticles;

(6) Sodium EDTA, Aspartame, Sunnett, Nipagin M/Nipasol M and FD&C Red 40 were completely dissolved in water to form a sweetening solution;

(7) hydroxypropyl methylcellulose having a methoxy content of 29% hydroxypropyl content of 8.5% and a viscosity (2%) of 4-6 cps (namely Methocel E15 commercially available from Dow Chemical Company) was wetted and uniformly mixed and uniformly mixed with ethanol, peppermint and strawberry twist, propylene glycol and cremophore;

(8) the sweetening solution of (6) was gradually added to the product of (7) with agitation until a homogenous viscous solution was obtained;

(9) the product of (5) was then added to the product of (8) with gentle agitation to form a final coating solution;

(10) the final coating solution of (9) was degassed, cast and dried to form opaque Famotidine containing intaoril film.

The Famotidine and Eudragit L-100 microparticles were present in the product film at a 1:2 ratio by weight to the sodium bicarbonate therein.

### Example 2

Another oral drug delivery device comprising bitterless Famotidine oral drug complex microparticals incorporated into a mucoadhesive intraoral film was prepared as follows:

(1) the polymethylacrylate copolymer was dissolved in ethanol;

(2) the Famotidine was suspended in sodium bicarbonated;

(3) the products of (1) and (2) were simultaneously pumped into a high energy ultrasonic processor, producing a complex solution in situ;

(4) the product of (3) was discharged to a spray dryer for drying, i.e. removal of the ethanol;

(5) the product of (4) was free-flowing, bitterless drug-polymer complex microparticles;

(6) Sodium EDTA, Aspartame, Sunnett, Nipagin M/Nipasol M and FD&C Red 40 were completely dissolved in water to form a sweetening solution;

(7) hydroxypropyl methylcellulose having a methoxy content of 29% hydroxypropyl content of 8.5 % and a viscosity (2%) of 4-6 cps (namely Methocel E15 commercially available from Dow Chemical Company) was wetted and uniformly mixed and uniformly mixed with ethanol, peppermint and strawberry twist, 70% sorbitol solution and cremophore;

(8) the sweetening solution of (6) was gradually added to the product of (7) with agitation until a homogenous viscous solution was obtained;

(9) the product of (5) was then added to the product of (8) with gentle agitation to form a final coating solution;

(10) the final coating solution of (9) was degassed, cast and dried to form opaque Famotidine containing intaoril film.

The Famotidine and Eudragit L-100 microparticles are present in the product film at a 1:2 ratio to the sodium bicarbonate therein.

### Example 3

Another oral drug delivery device comprising bitterless Famotidine oral drug complex microparticals incorporated into a mucoadhesive intraoral film was prepared as follows:

(1) the polymethylacrylate copolymer was dissolved in ethanol;

(2) the Famotidine was suspended in sodium bicarbonated;

(3) the products of (1) and (2) were simultaneously pumped into a high energy ultrasonic processor, producing a complex solution in situ;

(4) the product of (3) was discharged to a spray dryer for drying, i.e. removal of the ethanol;

(5) the product of (4) was free-flowing, bitterless drug-polymer complex microparticles;

(6) Sodium EDTA, Aspartame, Sunnett, Nipagin M/Nipasol M and FD&C Red 40 were completely dissolved in water to form a sweetening solution;

(7) hydroxypropyl methylcellulose having a methoxy content of 29% hydroxypropyl content of 8.5% and a viscosity (2%) of 4-6 cps (namely Methocel E15 commercially available from Dow Chemical Company) was wetted and uniformly mixed and uniformly mixed with ethanol, peppermint and strawberry twist, propylene glycol and cremophore;

(8) the sweetening solution of (6) was gradually added to the product of (7) with agitation until a homogenous viscous solution was obtained;

(9) the product of (5) was then added to the product of (8) with gentle agitation to form a final coating solution;

(10) the final coating solution of (9) was degassed, cast and dried to form opaque Famotidine containing intaoril film.

The Famotidine and Eudragit L-100 microparticles are present in the product film at a 1:2 ratio to the sodium bicarbonate therein.

### Example 4

An oral drug delivery device comprising bitterless Loratadine oral drug complex microparticals incorporated into a mucoadhesive intraoral film was prepared as follows:

(1) the polymethylacrylate copolymer was dissolved in ethanol;

(2) the Famotidine was suspended in sodium bicarbonated;

(3) the products of (1) and (2) were simultaneously pumped into a high energy ultrasonic processor, producing a complex solution in situ;

(4) the product of (3) was discharged to a spray dryer for drying, i.e. removal of the ethanol;

(5) the product of (4) was free-flowing, bitterless drug-polymer complex microparticles;

(6) Sodium EDTA, Aspartame, Sunnett, Nipagin M/Nipasol M and FD&C Red 40 were completely dissolved in water to form a sweetening solution;

(7) hydroxypropyl methylcellulose having a methoxy content of 29% hydroxypropyl content of 8.5% and a viscosity (2%) of 4-6 cps (namely Methocel E15 commercially available from Dow Chemical Company) was wetted and uniformly mixed and uniformly mixed with ethanol, peppermint and strawberry twist, 70% sorbitol solution, propylene glycol and cremophore;

(8) the sweetening solution of (6) was gradually added to the product of (7) with agitation until a homogenous viscous solution was obtained;

(9) the product of (5) was then added to the product of (8) with gentle agitation to form a final coating solution;

(10) the final coating solution of (9) was degassed, cast and dried to form opaque Famotidine containing intaoril film.

The Famotidine and Eudragit L-100 microparticles are present in the product film at a 1:2 ratio to the sodium bicarbonate therein.

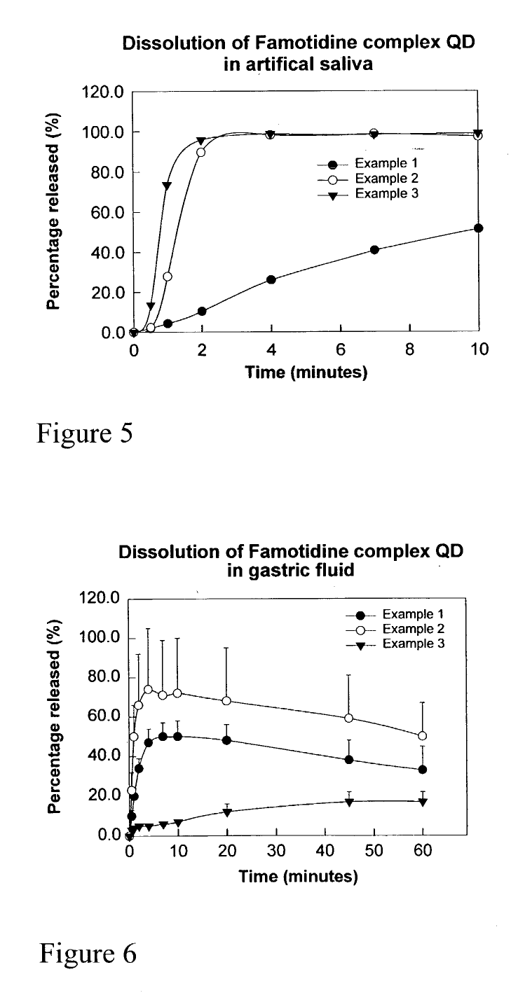

The dissolution profiles in artificial saliva of the oral drug delivery devices obtained according to Examples 1-3 were determined using USP apparatus, in 900 mL of dissolution media of artificial saliva solution, at rotation of 50 rpm, with a constant temperature bath at 37±0.5° C. Four-milliliter samples were drawn at 0.5, 1, 2, 4, 7, 10, 20, 45 and 60 minutes. The dissolution samples were filtered with a 0.45 m filter prior to analysis. The dissolution profile data obtained are presented in graphical form in FIG. 5.

The dissolution profiles in gastric fluid of the oral drug delivery devices obtained according to Examples 1-3 were determined using USP apparatus, in 900 mL of dissolution media of simulated gastric acid, at rotation of 50 rpm, with a constant temperature bath at 37±0.5° C. Four-milliliter samples were drawn at 0.5, 1, 2, 4, 7, 10, 20, 45 and 60 minutes. The dissolution samples were filtered with a 0.45 μm filter prior to analysis. The dissolution profile data obtained are presented in graphical form in FIG. 6.

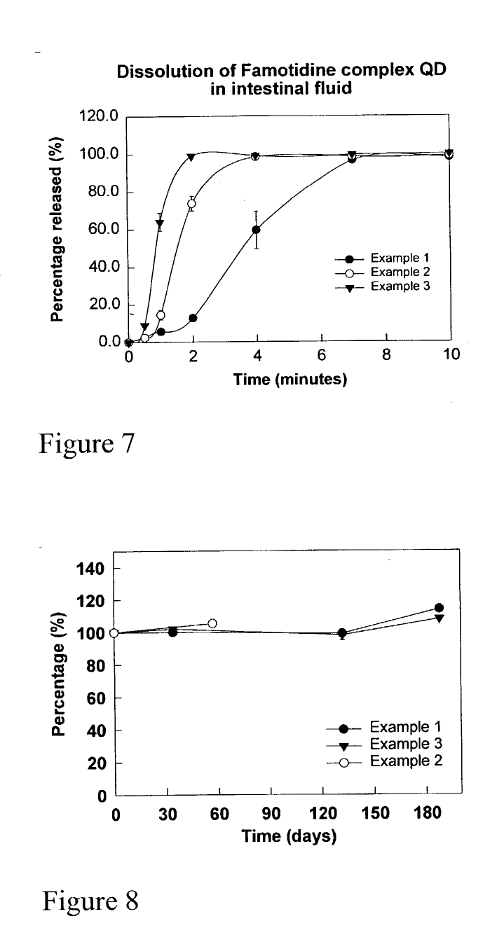

The dissolution profiles in intestinal fluid of the oral drug delivery devices obtained according to Examples 1-3 were determined using USP apparatus, in 900 mL of dissolution media of simulated intestinal fluid, at rotation of 50 rpm, with a constant temperature bath at 37±0.5° C. Four-milliliter samples were drawn at 0.5, 1, 2, 4, 7, 10, 20, 45 and 60 minutes. The dissolution samples were filtered with a 0.45 μm filter prior to analysis. The dissolution profile data obtained are presented in graphical form in FIG. 7.

The stability of the oral drug complex (Famotidine-Eudragit complex) in the oral drug delivery devices obtained according to Examples 1-3 was assessed using an HPLC to measure the drug content of the complex over time at ambient conditions. The results of the stability assessment are presented in graphical form in FIG. 8.

### Example 5

Oral drug complex microparticles according to the present invention containing Fomatodine and Eudragit L100 (commercially available from Rohm America Inc.) at a 1:1 ratio by weight. Buffer solutions with different pH from 2 to 7 were prepared by using 0.1 molar citric acid and 0.2 molar disodium phosphate. 100 mg of complex powder was added to 15 mL of each tested buffer solution, mixed well by rolling up to 2 hour. Before sampling, each sample was left to stand on the bench for about 5 minutes, then supernatant was drawn and filtered and analyzed by HPLC. The results of this analysis are presented in graphical form in FIG. 4.

### Example 6

The turbidity of oral drug complex microparticles (Famotidine-Eudragit L100) was assessed in various ionic strength solutions having a pH of about 6.6. A stock buffer solution with pH about 6.6 and ionic strength of about 307 mmol/L was prepared by using monobasic and dibasic sodium phosphate. A series of buffer solutions with different ionic strength were made from the stock solution by diluting. About 9 mg of complex powder with Eudragit L100/Famotidine at a 2:1 ratio was added into each buffer solution, the mixtures were occasionally shaken gently to help the complex powder to disperse in the buffer solution. Five minutes later, the samples were placed on a roller until the complex powder was dissolved. At 5, 9, 12, 16 and 30 minutes, the roller was turned off and the turbidity of each sample was examined visually. The results of these turbidity assessments are presented in tabular form in Table 1. A higher turbidity provides an indication that comparatively more material is undissolved. The results of this experiment indicate that the solubility of the complex powder increases when the ionic strength of the medium in which it is dissolved increases.

| | Ionic Strength | | | | | | | | |
|---|---|---|---|---|---|---|---|---|---|
| Time | (mEqu/liter) | | | | | | | | |
| (min.) | 34 | 61 | 102 | 153 | 170 | 219 | 225 | 256 | 307 |
| 5 | ++++ | +++ | +++ | ++ | ++ | ++ | ++ | ++ | ++ |
| 9 | +++ | ++ | ± | − | ± | − | − | − | − |
| 12 | ++ | + | − | − | − | − | − | − | − |
| 16 | + | − | − | − | − | − | − | − | − |
| 30 | − | − | − | − | − | − | − | − | − |

+: Turbidity (suspension)
−: Clarity (solution)

The present invention having been disclosed in connection with the foregoing embodiments, additional embodiments will now be apparent to persons skilled in the art. The present invention is not intended to be limited to the embodiments specifically mentioned, and accordingly reference should be made to the appended claims rather than the foregoing discussion, to assess the spirit and scope of the present invention in which exclusive rights are claimed.

We claim:

1. An oral drug complex microparticle comprising: a pharmaceutical active having a nitrogen moiety and an anionic copolymer; wherein the oral drug complex microparticle resists dissolution or dissociation in saliva but rapidly dissociates in gastric acid and dissolves in intestinal fluid; wherein the oral drug complex microparticle exhibits an ionic strength of less than 102 mEq/liter.

2. The oral drug complex microparticle of claim 1, wherein the nitrogen moiety is selected from the group of acyclic amine, heterocyclic amine, amide, imine, imide and nitrile.

3. The oral drug complex microparticle of claim 1, wherein the pharmaceutical active comprises Famotidine.

4. The oral drug complex microparticle of claim 1, wherein the pharmaceutical active comprises Loratidine.

5. The oral drug complex micropartilce of claim 1, wherein the oral drug complex microparticle has a particle size of less than 50 μm.

6. The oral drug complex microparticle of claim 1, wherein the oral drug complex microparticle has a particle size of less than 25 μm.

7. The oral drug complex microparticle of claim 1, wherein the anionic polymer comprises an anionic methacrylic acid copolymer.

8. An oral drug delivery device comprising an oral drug complex microparticle; wherein the oral drug complex microparticle comprises: a pharmaceutical active having a nitrogen moiety and an anionic polymer; wherein the oral drug complex microparticle resists dissolution or dissociation in saliva but rapidly dissociates in gastric acid and dissolves in intestinal fluid; wherein the oral drug complex microparticle exhibits an ionic strength of less than 102 mEq/liter.

9. The oral drug delivery device of claim 8, wherein the drug delivery device comprises one of a powder, a chewable tablet, tablet, a fast melt sugar tablet, a lyophilized wafer, an intraoral paper wafer, a mucoadhesive film and a non-mucoadhesive film.

10. The oral drug delivery device of claim 8, further comprising at lease one of a buffer, a stabilizer, a taste modifying agent, a preservative, a coloring agent, a surfactant/wetting agent, a plasticizer and a water soluble film former.

11. The oral drug delivery device of claim 10, wherein the buffer comprises sodium bicarbonate.

12. The oral drug delivery device of claim 8, wherein the pharmaceutical active is selected from the group consisting of Famotidine and Loratidine and wherein the anionic polymer comprises an anionic methacrylic acid copolymer.

13. An oral drug delivery device comprising an oral drug complex microparticle comprising a pharmaceutical active having a nitrogen moiety and an anionic polymer; wherein the oral drug complex microparticle resists dissolution or dissociation in saliva but rapidly dissociates in gastric acid and dissolves in intestinal fluid; wherein the oral drug complex microparticle exhibits an ionic strength of less than 102 mEq/liter; and wherein the oral drug complex microparticle has a particle size less than 50 μm.

14. The oral drug delivery device of claim 13, wherein the pharmaceutical active is selected from the group consisting of Famotidine and Loratidine.

15. The oral drug delivery device of claim 13, wherein the anionic polymer comprises an anionic methacrylic acid copolymer.

16. A process for producing an oral drug complex microparticle, comprising:
(a) dissolving an anionic polymer in an alcohol or a hydro-alcoholic solution;
(b) dissolving or suspending a pharmaceutical agent having a nitrogen moiety in a buffer solution;
(c) simultaneously feeding the products of (a) and (b) into a high energy ultrasonic processor to produce a complex solution;
(d) discharging the complex solution to a spray dryer for drying; and,
(e) collecting the product oral drug complex microparticles.

17. The process of claim 16, wherein the products of (a) and (b) are exposed to ultrasonic energy in (c) having a frequency between 25 and 40 kHz.

18. The process of claim 16, wherein the products of (a) and (b) are exposed in (c) to at least one of mechanical and electromechanical action by the ultrasonic processor.

19. The process of claim 16, wherein the products of (a) and (b) are exposed in (c) to ultrasonic energy until the complex solution becomes clear.

20. The process of claim 16, wherein the products of (a) and (b) are exposed in (c) to ultasonic energy having a frequency of between 25 and 40 kHz/MHz until the complex solution becomes clear.

21. The process of claim 16, wherein the buffer solution comprises a carbonated buffer.

22. The process of claim 16, wherein the buffer solution comprises sodium bicarbonate.

23. A method of treating a patient comprising administering to the patient an oral drug delivery device of claim 9.
